(19) United States
(12) Patent Application Publication
(10) Pub. No.: US 2023/0139522 A1
(43) Pub. Date: May 4, 2023

(54) HIGH-FREQUENCY ELECTRODE FOR USE IN A HANDHELD SURGICAL DEVICE, ELECTRODE INSTRUMENT, AND RESECTOSCOPE

(71) Applicant: OLYMPUS WINTER & IBE GMBH, Hamburg (DE)

(72) Inventors: Christoph KNOPF, Stockelsdorf (DE); Christian BROCKMANN, Hollenstedt (DE); Jiri LAVICKA, Prerov (CZ); Martin HORN, Hamburg (DE)

(73) Assignee: OLYMPUS WINTER & IBE GMBH, Hamburg (DE)

(21) Appl. No.: 17/974,274

(22) Filed: Oct. 26, 2022

Related U.S. Application Data
(60) Provisional application No. 63/274,752, filed on Nov. 2, 2021.
(60) Provisional application No. 63/276,904, filed on Nov. 8, 2021.

(51) Int. Cl. A61B 18/14

(52) U.S. Cl. CPC A61B 18/14; A61B 2018/1407; A61B 2018/1465; A61B 2018/00982

(57) ABSTRACT

High-frequency electrodes for handheld surgical devices are used above all in urology in electrosurgical work in the bladder, prostate, and urethra. These electrodes are known to be manufactured from a platinum-iridium alloy. The costs of these materials have proven to be particularly disadvantageous. A high-frequency electrode, an electrode instrument, and a resectoscope for which the production costs are reduced and the reliability of the electrode during the treatment is ensured. This is achieved in that the high-frequency electrode consists of a platinum-tungsten alloy, in particular is produced therefrom.

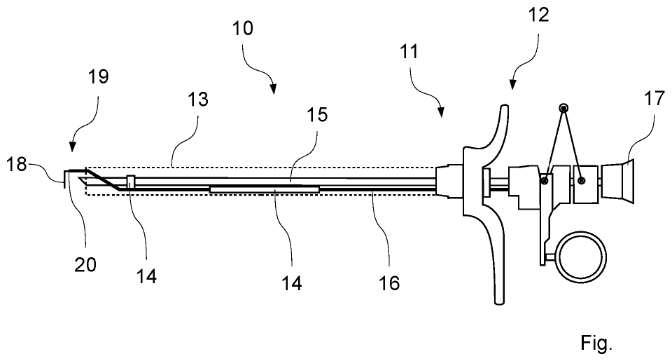

The invention relates to a high-frequency electrode for use in a handheld surgical device according to the preamble of claim 1. Furthermore, the invention relates to an electrode instrument for use in a handheld surgical device as claimed in claim 8 and to a resectoscope as claimed in claim 9.

High-frequency electrodes for handheld devices, in particular resectoscopes, of the generic type are used above all in urology in electrosurgical work in the bladder, prostate, and urethra. These high-frequency electrodes can also be used similarly for other surgical and orthopedic treatments and interventions. In general, these electrodes are used for resection and evaporation and electrocoagulation of tissue, for example of tissue in the lower urinary tract. For this purpose, the handheld devices or the resectoscopes comprise a generic high-frequency electrode or an electrode instrument which is mounted so it is longitudinally displaceable and rotatable within a shaft of the handheld device or of the resectoscope. The surgical high-frequency electrode is arranged at a distal working end of the electrode instrument.

Such electrodes can be made monopolar or bipolar. Depending on the application or embodiment, the electrodes are supplied with electrical energy or an electrical high-frequency voltage for this purpose via electrical conductors. The supply with the high-frequency current takes place via the handheld device or the resectoscope by a high-frequency generator. For the case where the electrode is designed as a monopolar electrode, a neutral electrode is arranged on the person to be treated. Alternatively, the neutral electrode can also be part of the resectoscope. In this case, the shaft, the transporter and the optical unit serve as the neutral electrode of an electrical potential. Alternatively, an electrode carrier can also and above all be used as neutral or return electrode. In particular with an extended electrode, this represents the shortest current path through the irrigation liquid.

Due to the application of a typical high-frequency voltage to the electrode, in a bipolar instrument, a plasma of electrically charged particles forms around the electrode or around some parts of the electrode. The plasma is localized directly at the electrode and has punctual a high temperature or energy density in spots.

High-frequency electrodes of the type in question are known to be manufactured from a platinum-iridium alloy. A long service life and at the same time a high level of mechanical stability of the electrode can be guaranteed by this alloy. This alloy made of the two noble metals platinum and iridium has proven to be particularly resistant to the main effects of degradation during plasma erosion. It has thus been shown that these electrodes have proven to be very resistant with respect to electrochemical oxidation, thermal evaporation, and with respect to atomic sputtering. Although the long lifetime of the material is of secondary importance in particular for the use as disposable electrodes, it has to be ensured in particular in the area of use described here that the high-frequency electrode functions perfectly at least over the duration of the treatment.

The costs of these materials have proven to be particularly disadvantageous. In particular the increased demand for the rare noble metal iridium has resulted in a significant increase in the costs. These increased material costs are also transferred to the production costs of the entire high-frequency electrode. A reduction of the iridium proportion in the electrode is not an option, since the functionality would thus be influenced to an excessive degree.

The invention is based on the object of providing a high-frequency electrode, an electrode instrument, and a resectoscope in which the production costs are reduced and the reliability of the electrode during the treatment is ensured.

One achievement of this object is described by the features of claim 1. It is accordingly provided that the high-frequency electrode for use in a handheld surgical device of the above-described type consists of a platinum-tungsten alloy, in particular is produced therefrom. A sufficiently high level of mechanical stability of the electrode may be achieved by this alloy. It can thus be ensured that the electrode also withstands greater mechanical forces during the treatment. The risk of breaking and the falling of fragments into the patient can thus be counteracted. A short circuit caused by excessively strong deformation of the electrode may also be avoided by the mentioned features. Moreover, this material selection has an elevated melting point due to the addition of tungsten. An extremely high resistance with respect to electrochemical oxidation and with respect to thermal evaporation can thus be achieved together with the noble metal platinum. Due to the combination of the noble metal platinum with the base metal tungsten, the degree of degradation by the ignition of the plasma will increase in comparison to the known platinum-iridium alloy, however, the defined procedure requirements are still exceeded for the above-mentioned types of treatment. Since tungsten is significantly less expensive to acquire and to handle than iridium, the costs for producing the high-frequency electrode may be reduced. It is therefore possible by way of the use of tungsten to meet the requirements for the high-frequency electrode and at the same time reduce the costs.

In particular, the invention provides that the tungsten proportion of the alloy is 5% to 12%. It is preferably also conceivable that the tungsten proportion is 9% or 11%. These proportional values of tungsten in the alloy have proven to be particularly advantageous for the functionality, the stability, and the resistance with respect to chemical and physical influences.

A further preferred exemplary embodiment of the invention provides that the loop of the electrode has a diameter of 0.25 mm to 0.35 mm, preferably of 0.27 mm to 0.30 mm. Furthermore, it is conceivable that the alloy from which the electrode is manufactured has a tensile strength of 1300 N/mm² to 1700 N/mm², preferably 1400 N/mm²-1600 N/mm², in particular 1500 N/mm²+/−50 N/mm². In addition, it is conceivable according to the invention that an elongation at fracture of the alloy is 2% to 10%, preferably 4% to 8%. It has been shown that this dimensioning and these mechanical properties of the alloy according to the invention are particularly suitable for carrying out the above-mentioned treatment steps and the required stability is thus achievable at the same time.

An electrode instrument for achieving the mentioned object has the features of claim 8. It is accordingly provided that the electrode instrument for use in a handheld surgical device, in particular in a resectoscope, has an elongate shaft section having two support arms, through which at least one conductor extends, which is connected or connectable at the distal end of the instrument to a high-frequency electrode as claimed in any one of claims 1 to 7. The high-frequency electrode is arranged between the distal ends of the support arms.

A resectoscope for achieving the object mentioned at the outset has the features of claim 9.

A preferred exemplary embodiment of the invention is described in more detail hereinafter on the basis of the single FIGURE of the drawing.

FIGURE. The FIGURE shows a schematic representation of a resectoscope with an indicated outer shaft.

A resectoscope 10 is shown as an example of a handheld surgical device in the FIGURE. This resectoscope 10 essentially consists of a transporter 11, a handle unit 12, and a shaft 13, which is to be guided into a corresponding body opening for the treatment of a patient. In the exemplary embodiment shown here, the shaft 13 is composed of an outer shaft tube, an optical unit 15, and an electrode instrument 16. The optical unit 15 consists of a long tube, in which lenses or glass fibers can be arranged to observe the region of the treatment at the distal end 19 of the shaft 13 through an eyepiece 17 arranged proximally on the shaft 13. Reference is made to the known prior art for a more detailed description of a resectoscope.

The electrode instrument 16 is essentially composed of a high-frequency electrode 18 and at least one electrical conductor 20. The at least one electrical conductor 20, on the one hand, supplies the high-frequency electrode 18 with a high-frequency voltage and, on the other hand, the conductor 20 and possibly a further element serves as a mount of the electrode 18 on the electrode instrument 16. The electrical conductor 20 leads from the distal end 19 of the resectoscope 10 through the shaft 13 and is connected via further lines to a high-frequency generator (not shown) for generating the high-frequency electromagnetic energy. It is conceivable here that the at least one conductor 20 of the electrode instrument 16 is fixed by mounts 14 inside the shaft 13, preferably on the optical unit 15.

Body tissue may be manipulated by means of the high-frequency electrode 18, for example. For this purpose, the high-frequency electrode 18 can be designed either as a monopolar or bipolar electrode. In the case of a bipolar electrode, it is connected to two electrical conductors 20. In the exemplary embodiment of a monopolar electrode, the electrode 18 is only connected to one electrical conductor 20. A further neutral electrode is attached to the patient or is integrated in the resectoscope. By applying electrical energy to the high-frequency electrode 18, a plasma is generated at the electrode 18, by means of which tissue is manipulated by a corresponding movement of the electrode instrument 16.

The high-frequency electrode 18 according to the present invention is formed as a loop or cutting loop. However, the high-frequency electrode 18 can also be formed as a knob, bar, strip, pin, or the like. This high-frequency electrode 18 is produced from a special alloy to have, for the above-described high-frequency applications, both sufficient resistance with respect to chemical and physical influences and also to be durable with respect to mechanical forces. According to the concept of the invention, the high-frequency electrode 18 consists of a platinum-tungsten alloy. This alloy has the advantage over the known alloys made of platinum and iridium that the material acquisition costs are significantly lower and in addition the production of the electrode is less complex and less costly. Moreover, a high-frequency electrode 18 may also be implemented by this alloy which at least fulfils the necessary process requirements.

1. A high-frequency electrode for use in a handheld surgical device, wherein the high-frequency electrode can be supplied with electrical energy via at least one electrical conductor, wherein the high-frequency electrode consists of a platinum-tungsten alloy.

2. The high-frequency electrode as claimed in claim 1, wherein the tungsten proportion of the alloy is 5% to 12%.

3. The high-frequency electrode as claimed in claim 1, wherein the tungsten proportion of the alloy is 9% to 11%.

4. The high-frequency electrode as claimed in claim 1, wherein the high-frequency electrode is formed as a loop, strip, or pin.

5. The high-frequency electrode as claimed in claim 4, wherein the loop has a wire diameter of 0.25 mm to 0.35 mm.

6. The high-frequency electrode as claimed in claim 1, wherein the alloy has a tensile strength of 1300 N/mm² to 1700 N/mm².

7. The high-frequency electrode as claimed in claim 1, wherein an elongation at fracture of the alloy is 2% to 10%.

8. An electrode instrument, for use in a handheld surgical device, wherein the electrode instrument has an elongate shaft section having two support arms, through which at least one conductor extends, which at a distal end of the electrode instrument forms a high-frequency electrode as claimed in claim 1 to which high-frequency current can be applied, and which is arranged between the distal ends of the support arms.

9. A resectoscope having an electrode instrument as claimed in claim 8.
